(19) United States
(12) Patent Application Publication
(10) Pub. No.: US 2005/0212953 A1
(43) Pub. Date: Sep. 29, 2005

(54) Piezoelectric focusing apparatus and method for the same

(76) Inventors: Alex Kuo, Hsin Chu City (TW); Franklin Lin, Taipei City (TW); Yu-Yao Chang, Taipei City (TW); Der-Jung Chen, Taipei City (TW)

Correspondence Address: BIRCH STEWART KOLASCH & BIRCH, PO BOX 747, FALLS CHURCH, VA, 22040-0747, US

(21) Appl. No.: 10/806,222

(22) Filed: Mar. 23, 2004

(51) Int. Cl. H04N 5/232; G03B 13/00; A61B 5/05; G03B 3/00; G03B 13/22; G03B 3/10; G03B 13/34

(52) U.S. Cl. 348/357; 396/93; 396/133

(57) ABSTRACT

A piezoelectric focusing apparatus and method for the same are proposed to quickly adjust a distance between a lens unit and an electronic imaging device. An expanding table associated with increased voltage and a shrinkage table associated with decreased voltage for the piezoelectric material is constructed. A bi-directional deformation table is then established by associating voltages on the expanding table and the shrinkage table corresponding to the same deformation. A voltage is supplied to the piezoelectric material according to the bi-directional deformation table for generating a desired deformation and controlling a focusing distance between the lens unit and the electronic imaging device.

### FIELD OF THE INVENTION

The present invention relates to a piezoelectric focusing apparatus and method for the same, and especially to a piezoelectric focusing apparatus for a digital imaging device such as digital still camera and method for the same.

### BACKGROUND OF THE INVENTION

With the rapid progress of digital electronics and semiconductor processes, many conventional consumer products have already been digitalized. For example, digital imaging devices such as digital still cameras (DSC) and digital video cameras (DV) are becoming increasingly mature and popular.

The digital imaging device uses an electronic imaging device such as a CCD (charge-coupled device) or a CMOS (complementary metal oxide semiconductor) sensor instead of conventional films to obtain an image. Moreover, the focusing unit thereof is also essential for the performance of the digital imaging device.

The present digital imaging device generally uses a lens unit controlled by a step motor to control the focusing of the image on the electronic imaging device. The step motor controls the distance between the lens unit and the electronic imaging device for adjusting focus based on a digital signal. However, the step motor has the drawbacks of a bulky size, of a low speed and of a high cost.

Alternatively, piezoelectric material such as piezoelectric ceramic can be used as a focusing unit for digital imaging device. Such a piezoelectric material produces a voltage signal due to mechanical deformation, and on the other hand, produces a mechanical deformation due to the voltage signal. Therefore, the distance between the lens unit and the electronic imaging device can be quickly adjusted. The distance for focusing adjustment is generally a minute value. Therefore, the piezoelectric focusing unit is feasible and has the advantages of a small volume, of a fast speed and of a low cost.

However, displacement control of the piezoelectric material is a non-linear analog control; in other words, when a linear voltage signal is supplied to the piezoelectric material, then it can be found that the proportion of the deformation of the piezoelectric material to the voltage signal is not geometrically equal; that is, as shown in the FIGS. 1A and 1B, both of the figures show a relationship curve between the voltage and the deformation of the piezoelectric material.

Because such a piezoelectric material usually has a hysteresis deformation property, the piezoelectric material has two different paths due to increasing of the voltage signal and due to decreasing of the voltage signal. As shown in FIG. 1A, in an expansion operation of the piezoelectric material, when the piezoelectric material is driven by a voltage that is increased from voltage V1 to voltage V2 along a forward path (curve C for expansion), then the piezoelectric material is deformed from deformation D1 to deformation D2. On the contrary, as shown in FIG. 1B, in a shrinkage operation of the piezoelectric material, when the piezoelectric material is driven by a voltage that is decreased from voltage V2 to voltage V1 along a backward path (curve D for shrinkage) different from the forward path, then the piezoelectric material is deformed from deformation D2 to deformation D1.

Therefore, in the conventional technology, in order to control the deformation of the piezoelectric material precisely, we must prepare two sets of deformation-voltage lookup table, one of which is used for expansion condition (voltage increasing control) and the other of which is used for shrinkage condition (voltage decreasing control). Therefore, the piezoelectric focusing apparatus can be driven to increase or decrease the distance between the lens unit and the electronic imaging device for adjusting focus.

However, a certain difficulty arises for the operation of the piezoelectric material with hysteretic response curve. Provided that the piezoelectric material is operated according to expansion curve C at the beginning and the deformation needs reduction, the piezoelectric material is firstly driven to top extreme D2 along the expansion curve C, then driven to bottom extreme D1 along the shrinkage curve D, and finally driven to a certain deformation, again, along expansion curve C.

Provided that the piezoelectric material is operated at shrinkage curve D at the beginning and the deformation needs to increase, the piezoelectric material is firstly driven to bottom extreme D1 along the shrinkage curve D, then driven to top extreme D2 along the expansion curve C, and finally driven to certain deformation again along shrinkage curve D. As can be seen from above description, the conventional piezoelectric focusing apparatus spend much time in the procedures and thus, cannot focus quickly.

### SUMMARY OF THE INVENTION

It is the object of the present invention to provide a piezoelectric focusing apparatus and method instead of the conventional step motor for controlling the distance between a lens unit and an electronic imaging device, in which the piezoelectric focusing apparatus and method can focus quickly.

To achieve the above object, the present invention provides the piezoelectric focusing method, in which a piezoelectric material is controlled to adjust the distance, between the lens unit and the electronic imaging device. A first table associated with increased voltage and a second table associated with decreased voltage for the piezoelectric material is constructed. A bidirectional deformation table is constructed by associating voltages on the first table and the second table corresponding to a deformation. A voltage is supplied to the piezoelectric material according to the bidirectional deformation table for generating a desired deformation and controlling a focusing distance between the lens unit and the electronic imaging device.

To achieve the above object, the present invention provides the piezoelectric focusing apparatus. The piezoelectric focusing apparatus comprises the electronic imaging device; at least one lens arranged- on one side of the electronic imaging device; a piezoelectric material placed between the lens and the electronic imaging device and used for adjusting a distance between the lens unit and the electronic imaging device; and a controller electrically connected to the piezoelectric material and having a built-in bidirectional deformation table. The controller supplies a voltage to the piezoelectric material according to the bidirectional deformation table for generating a desired deformation and controlling the focusing distance between the lens unit and the electronic imaging device.

The various objects and advantages of the present invention will be more readily understood from the following detailed description when read in conjunction with the appended drawing, in which:

### BRIEF DESCRIPTION OF DRAWING

The foregoing aspects and many of the attendant advantages of this invention will be more readily appreciated as the same becomes better understood by reference to the following detailed description, when taken in conjunction with the accompanying drawings, wherein:

FIGS. 1A and 1B show voltage-deformation relationship curve of the piezoelectric material.

FIG. 2 shows a schematic diagram of a preferred embodiment according to the present invention;

FIG. 3 shows the flowchart of the piezoelectric focusing method according to the present invention; and

FIG. 4 shows the operation of the piezoelectric focusing method according to the present invention.

### DETAILED DESCRIPTION OF THE INVENTION

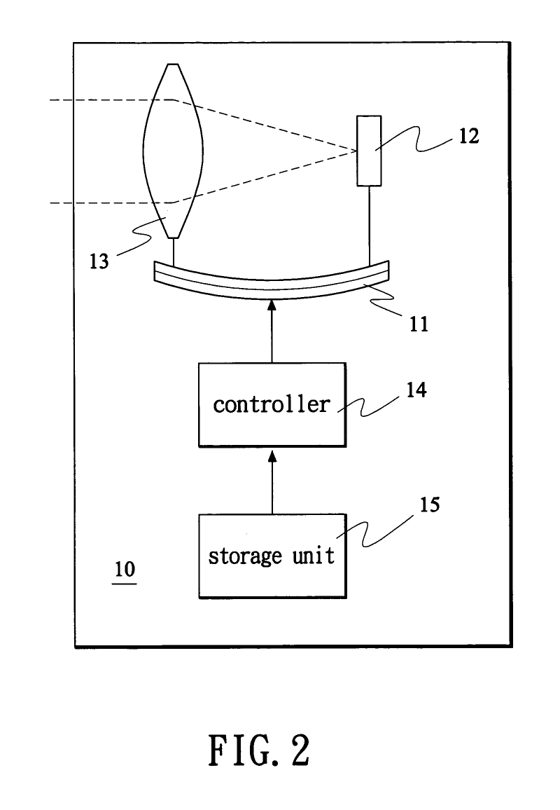

FIG. 2 shows a schematic diagram of a preferred embodiment according to the present invention. A digital imaging device 10 such as digital still camera (DSC), digital video camera (DV) or scanner is used for demonstration purpose. The digital imaging device 10 comprises a piezoelectric material 11, an electronic imaging device 12 such as a CCD (charge coupled device) or a CMOS sensor, a lens unit 13 for focusing the image on the electronic imaging device 12, a controller 14 and a storage unit 15.

The piezoelectric material 11 is arranged between the lens unit 13 and the electronic imaging device 12 for controlling the distance therebetween. The deformation of the piezoelectric material 11 is controlled by an applied voltage to control the focusing distance between the lens unit 13 and the electronic imaging device 12.

The controller 14 is electrically connected to the piezoelectric material 11 to control the deformation of the piezoelectric material 11 and is electrically connected to the storage unit 15 for fetching data needed to control the deformation of the piezoelectric material 11. The storage unit 15 stores a bi-directional deformation table, with which the controller 14 controls the deformation of the piezoelectric material 11, thus controlling the focusing distance between the lens unit 13 and the electronic imaging device 12.

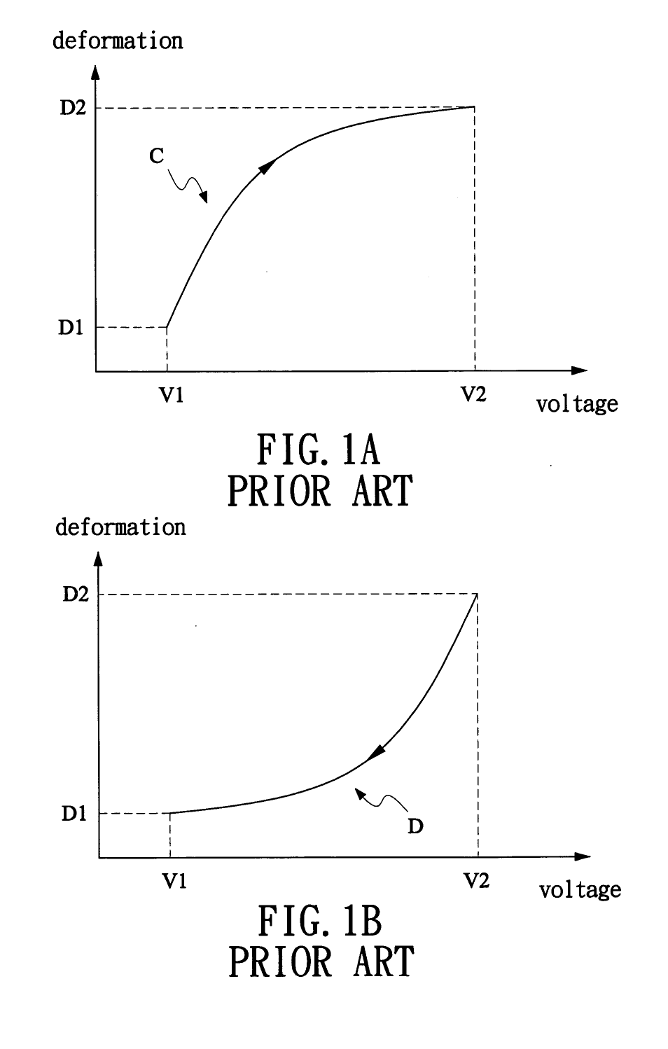

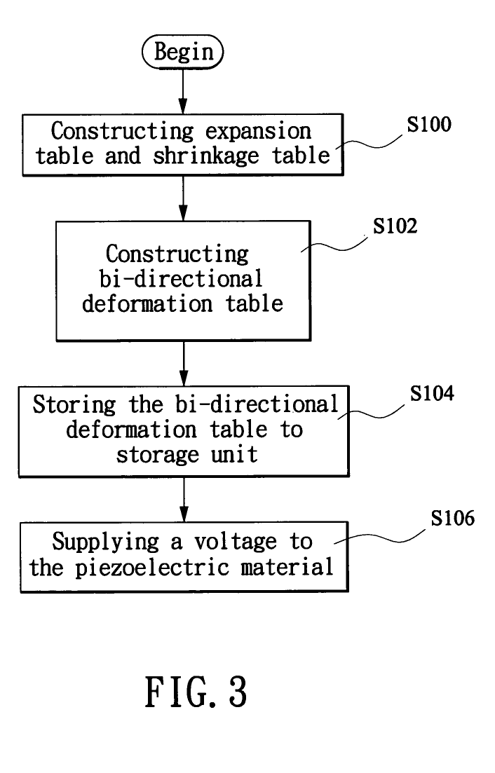

FIG. 3 shows the flowchart of the piezoelectric focusing method according to the present invention. In step S100, an expansion table corresponding to curve C (increasing voltage) and a shrinkage table corresponding to curve D (decreasing voltage) as shown in FIGS. 1A and 1B are prepared.

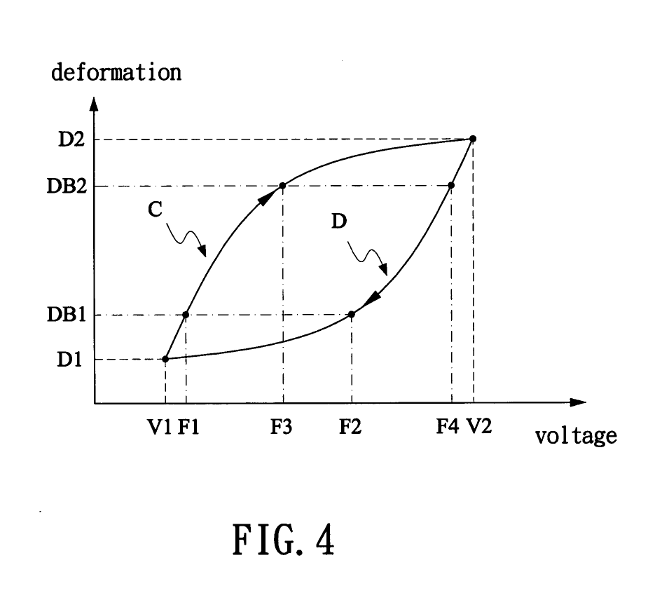

In step S102, a bi-directional deformation table is constructed by associating the same deformation appearing in the expansion table (increasing voltage) and the shrinkage table (decreasing voltage) but corresponding to different voltages as shown in FIG. 4. Then, in step S104, the bi-directional deformation table is stored in the storage unit 15.

Finally, the controller 14 supplies a suitable voltage to the piezoelectric material 11 according to the data stored in the bi-directional deformation table for expansion or shrinkage. Therefore, the focusing distance between the lens unit 13 and the electronic imaging device 12 can be controlled.

In step S100, the expansion table (increasing voltage) is constructed by increasing supplied voltage from V1 associated with minimal deformation D1 to V2 associated with maximal deformation D2, as depicted by the curve oriented from the vertex D1-V1 to vertex D2-V2 in FIG. 4.

In step S100, the shrinkage table (decreasing voltage) is constructed by decreasing supplied voltage from V2 associated with maximal deformation D2 to V1 associated with minimal deformation D1 as depicted by the curve oriented from the vertex D2-V2 to the vertex D1-V1 in FIG. 4.

The bidirectional deformation table is constructed by associating the voltages on the expansion table and the shrinkage table corresponding to the same deformation. For example, the voltage F1 on expansion table corresponds to deformation DB1 and the voltage F2 in the shrinkage table also corresponds to the same deformation DB1. Moreover, the voltage F3 in the expansion table corresponds to deformation DB2 and the voltage F4 in the shrinkage table also corresponds to the same deformation DB2.

FIG. 4 demonstrates the operation of the piezoelectric focusing method according to the present invention. Provided that the piezoelectric material 11 is initially operated according to the expansion curve C and the deformation is to be increased from DB1 to DB2, the controller 14 increases the supplied voltage from F1 to F2.

In the case where the piezoelectric material 11 is initially operated according to the shrinkage curve D and the deformation is to be increased from DB1 to DB2, the conventional piezoelectric focusing apparatus performs a complex procedure. In the complex procedure, the deformation is changed from DB1 to D1 by changing voltage from F2 to V1, the deformation is changed from D1 to D2 by changing voltage from V1 to V2, and then the deformation is changed from D12 to DB2 by changing voltage from V2 to F4.

According to the present invention, in the case where the piezoelectric material 11 is initially operated according to the shrinkage curve D and the deformation is to be increased from DB1 to DB2, the controller 14 performs a simple procedure. In the simple procedure, the deformation is changed from DB1 to D1 by changing the voltage from F2 to V1, and the deformation is changed from D1 to DB2 by changing the voltage from V1 to F3 according to the established bidirectional deformation table. Therefore, the piezoelectric focusing method can speed up the focusing operation.

Although the present invention has been described with reference to the preferred embodiment thereof, it will be understood that the invention is not limited to the details thereof. Various substitutions and modifications have suggested in the foregoing description, and other will occur to those of ordinary skill in the art. Therefore, all such substitutions and modifications are intended to be embraced within the scope of the invention as defined in the appended claims.

1. A piezoelectric focusing method, a piezoelectric material being controlled to adjust a distance between a lens unit and an electronic imaging device, the method comprising the steps of:
constructing a first table associated with an increased voltage and a second table associated with a decreased voltage for the piezoelectric material;
constructing a bi-directional deformation table by associating voltages in the first table and the second table corresponding to a deformation; and
supplying a voltage to the piezoelectric material according to the bi-directional deformation table for generating a desired deformation and controlling a focusing distance between the lens unit and the electronic imaging device.

2. The piezoelectric focusing method as in claim 1, wherein the piezoelectric material is a deformable material with hysteretic characteristic.

3. The piezoelectric focusing method as in claim 1, wherein the piezoelectric material is expanded or shrunk according to an applied voltage thereon.

4. The piezoelectric focusing method as in claim 1, wherein the electronic imaging device is a CCD (charge coupled device) or a CMOS sensor.

5. The piezoelectric focusing method as in claim 1, wherein the step of constructing the first table is performed by increasing a supplied voltage from one associated with a minimal deformation to another associated with a maximal deformation.

6. The piezoelectric focusing method as in claim 1, wherein the step of constructing a second table is performed by decreasing a supplied voltage from one associated with a maximal deformation to another associated with a minimal deformation.

7. The piezoelectric focusing method as in claim 1, wherein the step of constructing the bi-directional deformation table is performed by associating voltages on the first table related to an expanding operation and the second table related to a shrinking operation corresponding to a same deformation.

8. The piezoelectric focusing method as in claim 1, further, after the step of constructing the bi-directional deformation table, comprising a step of
storing the bi-directional deformation table.

9. The piezoelectric focusing method as in claim 1, wherein the step of supplying a voltage to the piezoelectric material according to the bi-directional deformation table is performed for expanding and shrinking.

10. A piezoelectric focusing apparatus, comprising:
an electronic imaging device;
at least one lens arranged on one side of the electronic imaging device;
a piezoelectric material placed between the lens and the electronic imaging device and used for adjusting a distance between the lens unit and the electronic imaging device; and
a controller electrically connected to the piezoelectric material and having a built-in bi-directional deformation table, the controller supplying a voltage to the piezoelectric material according to the bidirectional deformation table for generating a desired deformation and controlling a focusing distance between the lens unit and the electronic imaging device.

11. The piezoelectric focusing apparatus as in claim 10, wherein the electronic imaging device is a CCD (charge coupled device) or a CMOS sensor.

12. The piezoelectric focusing apparatus as in claim 10, wherein the piezoelectric material is a deformable material with a hysteretic characteristic.

13. The piezoelectric focusing apparatus as in claim 10, wherein the piezoelectric material is expanded or shrunk according to voltage applied thereon.

14. The piezoelectric focusing apparatus as in claim 10, wherein the bi-directional deformation table is constructed by associating voltages on a first table related to expanding operation and a second table related to shrinking operation corresponding to a same deformation.

15. The piezoelectric focusing apparatus as in claim 10, further comprising a storage unit electrically connected to the controller and used for storing the bi-directional deformation table.
